(19) United States
(12) Patent Application Publication
(10) Pub. No.: US 2002/0087106 A1
(43) Pub. Date: Jul. 4, 2002

(54) Wound dressing

(76) Inventors: Paul D. Unger, St. Catherines (CA); Victor Unger, St. Catherines (CA)

Correspondence Address: LINGBECK PATENT LAW OFFICE, 380 JACKSON STREET, SUITE 417, ST. PAUL, MN, 55101, US

(21) Appl. No.: 10/084,080

(22) Filed: Feb. 27, 2002

(51) Int. Cl. A61F 13/00

(52) U.S. Cl. 602/48; 602/54; 602/57

(57) ABSTRACT

A wound dressing for facilitating the healing of a wound. The wound dressing includes a support strip of material having a body-attaching side; and also having adhesive disposed upon the body-attaching side; and also includes a pad member being securely attached upon the body-attaching side of the support strip of material; and further includes at least one wound-healing agent being disposed upon the pad member; and also includes protective strips of material being removably disposed over the body-attaching side including the pad member and the at least one wound-healing agent.

### BACKGROUND OF THE INVENTION

1. Field of the Invention

The present invention relates to adherable wound dressings and more particularly pertains to a new wound dressing for facilitating the healing of a wound.

2. Description of the Prior Art

The use of adherable wound dressings is known in the prior art. More specifically, adherable wound dressings heretofore devised and utilized are known to consist basically of familiar, expected and obvious structural configurations, notwithstanding the myriad of designs encompassed by the crowded prior art which have been developed for the fulfillment of countless objectives and requirements.

Known prior art includes U.S. Pat. No. 6,171,604; U.S. Pat. No. 4,808,172; U.S. Pat. No. 5,931,800; U.S. Pat. No. Des. 424,699; U.S. Pat. No. 6,099,866; U.S. Pat. No. 6,156,323; and U.S. Pat. No. 6,156,334.

While these devices fulfill their respective, particular objectives and requirements, the aforementioned patents do not disclose a new wound dressing. The prior art describes inventions including strips of material having gauze centrally-disposed thereupon.

### SUMMARY OF THE INVENTION

The general purpose of the present invention, which will be described subsequently in greater detail, is to provide a new wound dressing which has many of the advantages of the adherable wound dressings mentioned heretofore and many novel features that result in a new wound dressing which is not anticipated, rendered obvious, suggested, or even implied by any of the prior art adherable wound dressings, either alone or in any combination thereof. The present invention includes a support strip of material having a body-attaching side; and also having adhesive disposed upon the body-attaching side; and also includes a pad member being securely attached upon the body-attaching side of the support strip of material; and further includes at least one wound-healing agent being disposed upon the pad member; and also includes protective strips of material being removably disposed over the body-attaching side including the pad member and the at least one wound-healing agent. None of the prior art describes using the particular type of wound-healing agents as described in the present invention.

There has thus been outlined, rather broadly, the more important features of the wound dressing in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

It is an object of the present invention to provide a new wound dressing which has many of the advantages of the adherable wound dressings mentioned heretofore and many novel features that result in a new wound dressing which is not anticipated, rendered obvious, suggested, or even implied by any of the prior art adherable wound dressings, either alone or in any combination thereof.

Still another object of the present invention is to provide a new wound dressing for facilitating the healing of a wound.

Still yet another object of the present invention is to provide a new wound dressing that is easy and convenient to use.

Even still another object of the present invention is to provide a new wound dressing that uses a natural substance to effect healing of a wound to a user's body.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of a new wound dressing according to the present invention.

FIG. 2 is an exploded front view of the present invention.

FIG. 3 is a longitudinal cross-sectional view of the strip of material of the present invention.

FIG. 4 is a front view of other embodiments of the present invention.

### DESCRIPTION OF THE PREFERRED EMBODIMENT

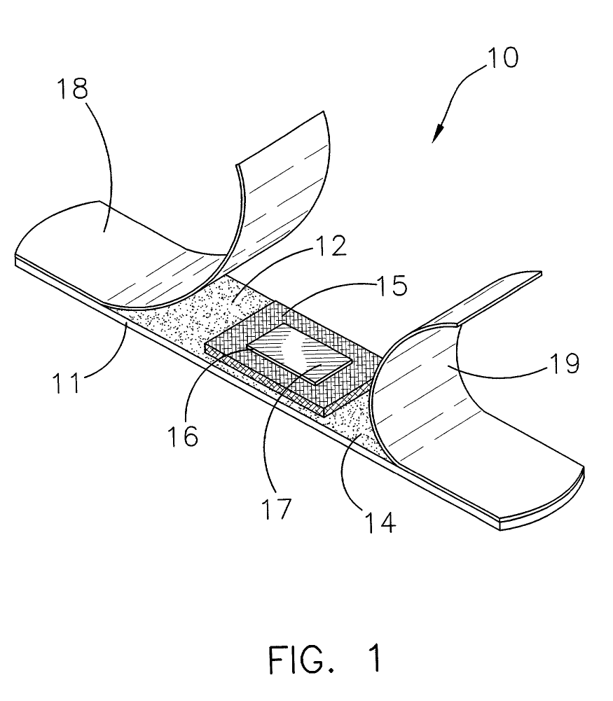

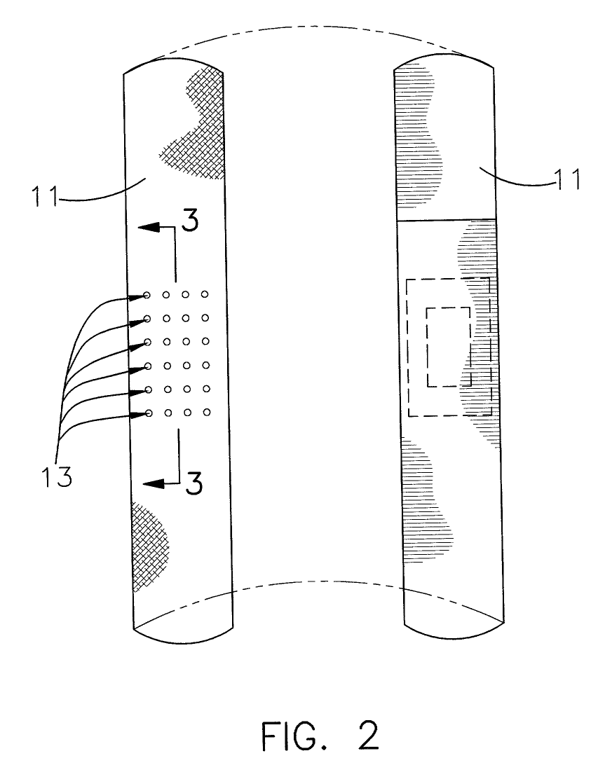

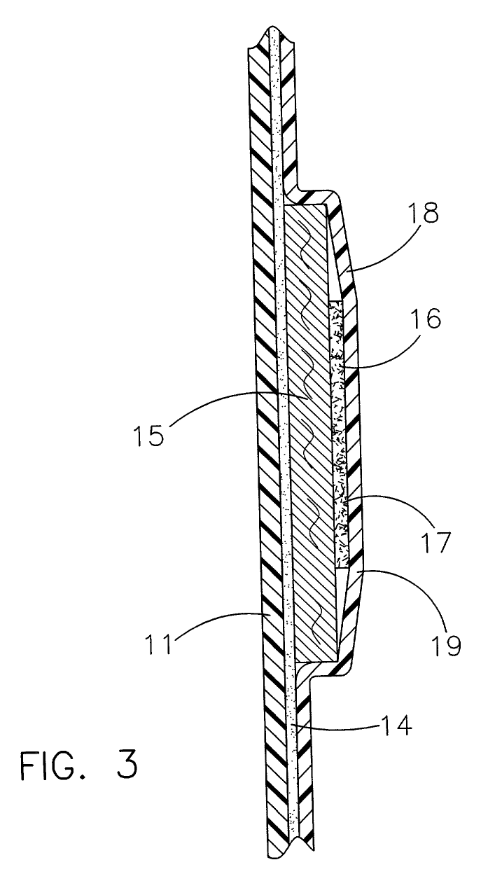

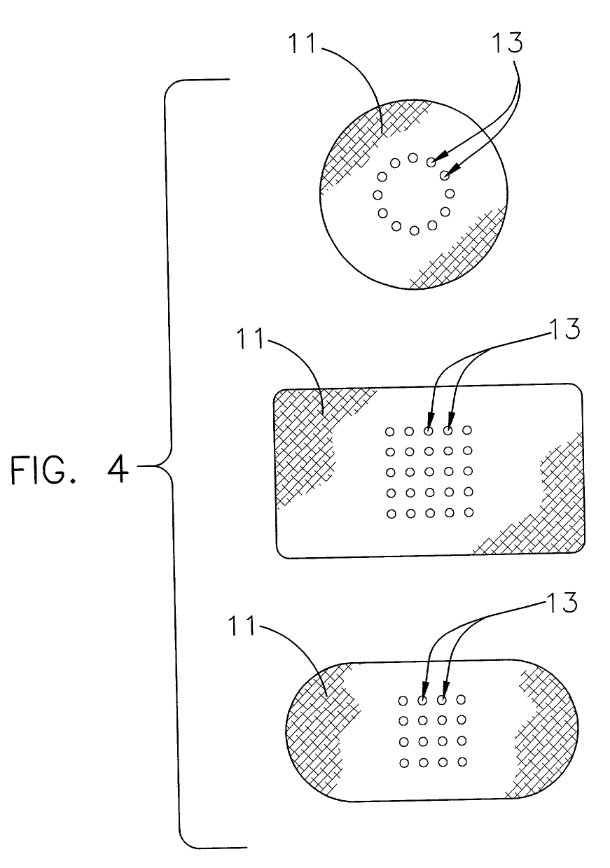

With reference now to the drawings, and in particular to FIGS. 1 through 4 thereof, a new wound dressing embodying the principles and concepts of the present invention and generally designated by the reference numeral 10 will be described.

As best illustrated in FIGS. 1 through 4, the wound dressing 10 generally comprises a support strip of material 11 having a body-attaching side 12; and also having adhesive 14 being conventionally disposed about and upon the body-attaching side 12. The support strip of material 11 is made of flexible plastic and has a plurality of aerating openings 13 being spaced about and being disposed through the support strip of material 11.

A pad member 15 is securely and conventionally attached upon the body-attaching side 12 of the support strip of material 11. The pad member 15 is generally a gauze and is centrally attached upon the body-attaching side 12 of the support strip of material 11 for covering a wound on a user's body. Wound-healing agents 16, 17 are conventionally disposed upon the pad member 15. The wound-healing agents include a syrupy substance known as honey 16, and also include a wound-sealing wax 17 being conventionally disposed upon the gauze 15 and being mixed with the honey 16.

Protective strips of material 18, 19 are removably disposed over the body-attaching side 12 including the pad member 15 and the wound-healing agents 16, 17. The protective strips of material 18, 19 are made of flexible fabric and have end portions which overlap one another when detachably attached to the support strip of material 11.

In use, the user removes the protective strips of material 18, 19 by grasping the end portions and pulling them off the support strip of material 11, and then applies the body-attaching side 12 of the support strip of material 11 directly upon the user's body with pad member 15 and wound-healing agents 16, 17 being placed upon the wound to effectively stop the bleeding and to effectively heal the wounded area of the body.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the wound dressing. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A wound dressing comprising:
a support strip of material having a body-attaching side; and also having adhesive being disposed about and upon said body attaching side;
a pad member being securely attached upon said body-attaching side of said support strip of material;
at least one wound-healing agent being disposed upon said pad member; and
protective strips of material being removably disposed over said body-attaching side including said pad member and said at least one wound-healing agent.

2. A wound dressing as described in claim 1, wherein said support strip of material is made of flexible plastic and has a plurality of aerated openings being spaced about and being disposed through said support strip of material.

3. A wound dressing as described in claim 1, wherein said pad member is generally a gauze and is centrally attached upon said body-attaching side of said support strip of material.

4. A wound dressing as described in claim 3, wherein said at least one wound-healing agent includes a syrupy substance known as honey.

5. A wound dressing as described in claim 4, wherein said at least one wound-healing agent also includes a wound-sealing wax being disposed upon said gauze along with said honey.

6. A wound dressing as described in claim 5, wherein said protective strips of material are made of flexible fabric and have end portions which overlap one another when detachably attached to said support strip of material.
